(19) United States
(12) Patent Application Publication
(10) Pub. No.: US 2020/0129633 A1
(43) Pub. Date: Apr. 30, 2020

(54) PHARMACEUTICAL COMPOSITION COMPRISING C-MET ANTIBODY-DRUG CONJUGATE AND USE THEREOF

(71) Applicants: Jiangsu Hengrui Medicine Co., Ltd., Lianyungang, Jiangsu (CN); Shanghai Hengrui Pharmaceutical Co., Ltd., Minhang District, Shanghai (CN)

(72) Inventors: Jingjing FANG, Minhang District, Shanghai (CN); Zhen YAN, Minhang District, Shanghai (CN); Guimei TONG, Minhang District, Shanghai (CN); Xun LIU, Minhang District, Shanghai (CN)

(21) Appl. No.: 16/618,974

(22) PCT Filed: Jun. 5, 2018
(86) PCT No.: PCT/CN2018/089955

§ 371 Date: Dec. 3, 2019

(30) Foreign Application Priority Data
Jun. 6, 2017 (CN) 201710417725.4
Jul. 7, 2017 (CN) 201710551046.6

(51) Int. Cl. A61K 47/68; C07K 16/28; A61K 9/19

(52) U.S. Cl. CPC A61K 47/6807; A61K 9/19; C07K 16/28

(57) ABSTRACT

A stable c-Met antibody-drug conjugate (c-Met ADC) formulation and use thereof in medicine are described. In particular, the formulation contains c-Met ADC, a buffer, and can also contains at least one stabilizer, and optionally a surfactant. The c-Met ADC formulation of the application can effectively inhibit the aggregation and isomerization of antibodies, and prevent the degradation of an antibody product therein, being a stable injectable pharmaceutical formulation.

### CROSS-REFERENCE TO RELATED APPLICATIONS

This application is a Section 371 of International Application No. PCT/CN2018/089955, filed Jun. 5, 2018, which was published in the Chinese language on Dec. 13, 2018 under International Publication No. WO 2018/223958 A1, which claims priority under 35 U.S.C. § 119(b) to Chinese Patent Application No. CN201710417725.4, filed on Jun. 6, 2017, and Chinese Patent Application No. CN201710551046.6 filed on Jul. 7, 2017, the contents of which are incorporated herein by reference in their entireties.

### FIELD OF THE INVENTION

The present disclosure relates to the field of pharmaceutical preparations, in particular to a pharmaceutical composition comprising a c-Met antibody drug conjugate and a use thereof as a medicament.

### BACKGROUND OF THE INVENTION

The c-Met proto-oncogene is located at the long arm of human chromosome 7 (7q31) and spans over 120 kb in length. It encodes a c-Met protein precursor with molecular weight of approximately 150 kD, which undergoes local glycosylation to form a 170 kD glycoprotein. The glycoprotein is further cleaved into an α-subunit (50 kDa) and a (3-subunit (140 kDa), which are linked by disulfide bonds to form a mature c-Met protein receptor. The heterodimer comprises two strands. The β chain has an extracellular region, a transmembrane region (also known as a membrane stretch fragment) and an intracellular region (including an intracellular tyrosine kinase binding site). The α chain has only an extracellular portion, but it is highly glycosylated and attached to the β chain via disulfide bonds. The extracellular region of the two subunits is the recognition site of the corresponding ligand, and the intracellular region has tyrosine kinase activity.

The mechanism of c-Met activation is classified into three types: (i) the activation mechanism dependent on HGF, (ii) the activation mechanism independent of HGF, and (iii) the activation through other membrane pathways, such as hyaluronic acid receptor CD44 on the membrane surface, adhesin, RON signal transduction pathway, etc. The most common of these is the activation mechanism dependent on HGF. The N-terminus of HGF binds to c-Met, promoting the dimerization and autophosphorylation of Tyr1234 and Tyr1235 on the β chain, and the phosphorylation of Tyr1349 and Tyr1356 adjacent to the C-terminus to produce a binding site for multiple adaptor proteins. These adaptor proteins induce the activation of downstream signals mediated by P13K/Akt, Ras/Mapk, c-Src and STAT3/5, triggering different cellular responses such as cell survival and activity (closely related to the P13K/Akt pathway), tumor metastasis and cell proliferation (mainly mediated by Ras/Mapk). In addition, c-Met has cross-talk with other membrane receptors, and the cross-talk has been known to promote tumor formation and metastasis. Since c-Met is the intersection of many pathways leading to tumor formation and metastasis, it is relatively easy to achieve simultaneous interference on multiple pathways by targeting c-Met, thus c-Met has become a promising target for anti-tumor and metastasis treatment.

Antibody drug conjugate (ADC) is formed by attaching a monoclonal antibody or antibody fragment to a biologically active cytotoxin via a stable chemical linker compound, making full use of the specificity of the antibody to tumor cells or highly expressed antigens and the high efficiency of the cytotoxin to avoid toxic side effects on normal cells. This means that antibody drug conjugates are capable of precisely binding to tumor cells and reducing the effects on normal cells compared to traditional chemotherapeutic drugs.

An ADC drug consists of an antibody (targeting moiety), a linker and a toxin, wherein a good targeting moiety determines the specificity of the ADC drug, which includes not only specific target binding, but also effective endocytosis.

However, antibody drugs, especially ADCs, are less stable than other chemical drugs because of their larger molecular weight, more complex structures, and susceptibility for degradation, aggregation, or undesired chemical modification. In order to make the antibody drug conjugates suitable for administration and maintain stability during storage and subsequent use, so as to exert better effects, stable preparations of antibody drugs are particularly important.

A number of companies are currently developing pharmaceutical preparations comprising c-Met antibodies or antibody drug conjugates, such as: CN103781493A, CN105050618A, WO2016042412A1, etc. However, regarding to c-Met ADC, these preparations are not optimal preparation compositions. The present disclosure provides a pharmaceutical composition (preparation) comprising a c-Met ADC which is sufficiently stable and more suitable for administration.

### SUMMARY OF THE INVENTION

The present disclosure provides a pharmaceutical composition comprising a c-Met antibody drug conjugate, and other excipient(s).

In some embodiments, provided herein is a pharmaceutical composition comprising a c-Met antibody drug conjugate and a buffer; the buffer is preferably a succinate buffer or a citrate buffer, more preferably a succinate buffer.

In some embodiments, in the pharmaceutical composition, the concentration of the c-Met antibody drug conjugate is 1 mg/mL to 30 mg/mL, preferably about 1 mg/mL to 20 mg/mL, further preferably about 5 mg/mL to 20 mg/mL, most preferably 10-20 mg/mL; non-limiting examples include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL and 30 mg/mL.

In some embodiments, the pH of the pharmaceutical composition is about 5.0 to 6.0, preferably about 5.0 to 5.5, most preferably 5.0, 5.1, 5.2, 5.3, 5.4 or 5.5.

In some embodiments, in the pharmaceutical composition, the concentration of the buffer is about 5 mM to 30 mM, preferably 5 mM to 20 mM, further preferably about 10 mM to 20 mM, more preferably about 10 mM to 15 mM, most preferably 10 mM.

In some embodiments, the pharmaceutical composition further comprises a saccharide. The “saccharide” comprises general compositions (CH₂O)ₙ and derivatives thereof, including monosaccharide, disaccharide, trisaccharide, polysaccharide, sugar alcohol, reducing sugar, non-reducing sugar, etc. The saccharide can be selected from the group consisting of glucose, sucrose, trehalose, lactose, fructose, maltose, dextran, glycerin, dextran, erythritol, glycerol, arabitol, sylitol, sorbitol, mannitol, melibiose, melezitose, raffinose, mannotriose, stachyose, maltose, lactulose, maltotriose, sorbitol, maltitol, lactitol, isomaltulose and the like. The saccharide is preferably a non-reducing disaccharide, more preferably trehalose or sucrose. The pharmaceutical composition further comprises a disaccharide; the disaccharide is preferably trehalose or sucrose, most preferably trehalose.

In some embodiments, in the pharmaceutical composition, the concentration of the saccharide is about 40 mg/mL to about 80 mg/mL, preferably 50 mg/mL to about 70 mg/mL, more preferably 55 mg/mL to about 65 mg/mL; non-limiting examples include 55 mg/mL, 57 mg/mL, 59 mg/mL, 60 mg/mL, 61 mg/mL, 63 mg/mL and 65 mg/mL.

In some embodiments, the pharmaceutical composition further comprises a surfactant, which can be selected from the group consisting of polysorbate 20, polysorbate 80, poloxamer, Triton, sodium dodecyl sulfate, sodium lauryl sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauramidopropyl-betaine, cocamidopropyl-betaine, linoleamidopropyl-betaine, myristamidopropyl-betaine, palmitamidopropyl-betaine, isostearamidopropyl-betaine, myristamidopropyl-dimethylamine, palmitamidopropyl-dimethylamine, isostearamidopropyl-dimethylamine, sodium methyl cocoyl, sodium methyl oleyl taurate, polyethylene glycol, polypropylene glycol, a copolymer of ethylene and propylene glycol, etc. The surfactant is preferably polysorbate 80 or polysorbate 20, more preferably polysorbate 20.

In some embodiments, the concentration of the surfactant in the pharmaceutical composition is about 0.05 mg/mL to 1.0 mg/mL, further preferably 0.05 mg/mL to 0.4 mg/mL, more preferably 0.1 mg/mL to 0.4 mg/mL, most preferably 0.1 mg/mL to 0.2 mg/mL; non-limiting examples include 0.1 mg/mL, 0.2 mg/mL, 0.3 mg/mL and 0.4 mg/mL.

Also provided herein is a pharmaceutical composition comprising:

(a) 1-20 mg/mL c-Met antibody drug conjugate;

(b) 10-20 mM succinate buffer, pH 5.0-5.5;

(c) 40-80 mg/mL α,α-trehalose dihydrate; and

(d) 0.05-0.4 mg/mL polysorbate 20.

In some embodiments, the pharmaceutical composition comprises: (a) 1-20 mg/mL c-Met antibody drug conjugate; (b) 10-20 mM succinate buffer, pH 5.0-5.5; (c) 60 mg/mL α,α-trehalose dihydrate; and (d) 0.05-0.4 mg/mL polysorbate 20.

In some embodiments, the pharmaceutical composition comprises: (a) 5-20 mg/mL c-Met antibody drug conjugate; (b) 10-20 mM succinate buffer, pH 5.0-5.5; (c) 50-70 mg/mL α,α-trehalose dihydrate; and (d) 0.1-0.2 mg/mL polysorbate 20.

In some embodiments, in the pharmaceutical composition, the antibody in the c-Met antibody drug conjugate is Ab-10; the heavy chain sequence of the Ab-10 antibody is shown as SEQ ID NO: 24 of WO2016/165580A1, and the light chain sequence of the Ab-10 antibody is shown as SEQ ID NO: 27 of WO2016/165580A1.

In some embodiments, in the pharmaceutical composition, the c-Met antibody drug conjugate is ADC-12, which has a structure as shown below:

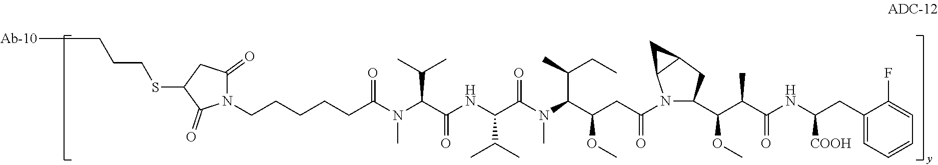

wherein, y ranges from 1 to 8, preferably from 2 to 5; y can be a decimal.

In some embodiments, the pharmaceutical composition comprises: (a) 1-30 mg/mL ADC-12; (b) 10-30 mM succinate buffer or citrate buffer, pH 5.0-5.5 (c) 40-80 mg/mL α,α-trehalose dihydrate; and (d) 0.05-0.4 mg/mL polysorbate 20.

In some embodiments, the pharmaceutical composition comprises: (a) 1-20 mg/mL ADC-12; (b) 10-20 mM succinate buffer, pH 5.0-5.5; (c) 40-80 mg/mL α,α-trehalose dihydrate; and (d) 0.05-0.4 mg/mL polysorbate 20.

In some embodiments, the pharmaceutical composition comprises: (a) 1-20 mg/mL ADC-12; (b) 10-20 mM succinate buffer, pH 5.0-5.5; (c) 60 mg/mL α,α-trehalose dihydrate; and (d) 0.05-0.4 mg/mL polysorbate 20.

In some embodiments, the pharmaceutical composition comprises: (a) 5-20 mg/mL ADC-12; (b) 10-20 mM succinate buffer, pH 5.0-5.5; (c) 50-70 mg/mL α,α-trehalose dihydrate; and (d) 0.1-0.2 mg/mL polysorbate 20.

In some embodiments, the pharmaceutical composition comprises: 1 mg/mL ADC-12 and 20 mM succinate buffer, pH 5.5 or 6.0.

In some embodiments, the pharmaceutical composition comprises: 1 mg/mL ADC-12 and 20 mM citrate buffer, pH 5.0, 5.5 or 6.0.

In some embodiments, the pharmaceutical composition comprises: 20 mg/mL ADC-12, 10 mM succinate buffer or citrate buffer, 60 mg/mL sucrose and 0.2 mg/mL polysorbate 20, pH 5.5.

In some embodiments, the pharmaceutical composition comprises: 20 mg/mL ADC-12, 10 mM succinate buffer, 60 mg/mL α,α-trehalose dihydrate and 0.05-0.4 mg/mL polysorbate 20, pH 5.0-5.5.

In some embodiments, the pharmaceutical composition comprises: 20 mg/mL ADC-12, 10 mM succinate buffer, 60 mg/mL α,α-trehalose dihydrate and 0.2 mg/mL polysorbate 20, pH 5.0-5.5.

In some embodiments, the pharmaceutical composition comprises: 20 mg/mL ADC-12, 10 mM succinate buffer, 60 mg/mL α,α-trehalose dihydrate and 0.2 mg/mL polysorbate 20, pH 5.3.

Also provided herein is a method of preparing a lyophilized preparation comprising c-Met antibody drug conjugate, wherein the method comprises lyophilizing the foregoing pharmaceutical composition.

In some embodiments, the method of preparing a lyophilized preparation comprising c-Met antibody drug conjugate comprises pre-freezing, primary drying and secondary drying in sequence. The purpose of pre-freezing is to freeze the product to obtain a crystalline solid. The pre-freezing temperature and the pre-freezing rate are two important process parameters. The optimal setting of the pre-freezing temperature and the pre-freezing rate of the present disclosure is −45° C. and 1° C./min (starting from −5° C.), respectively. Primary drying, also known as main drying, is the main stage of sample lyophilization. The purpose of primary drying is to remove ice from the product while maintaining shape of the product and minimizing damage to the product. If the temperature and the vacuum degree of primary drying are not proper, it will cause collapse of the product. Higher temperature and higher vacuum degree will accelerate the lyophilization, while the collapse risk of the product is increased at the meantime. The temperature of the primary drying of the present disclosure can be a temperature conventional in the art, for example, −27° C. to −20° C., preferably −20° C. Secondary drying, also known as desorption drying, is the main step of removing bound water from the product under ultimate vacuum (0.01 mbar) and higher temperature (25-40° C.). Since most biologics are sensitive to temperature, the secondary drying is conducted at a lower point of the temperature range, i.e. 25° C. In addition, the conditions of lyophilization can vary with the preparation, the size and the type of the sample container and the volume of the liquid during practical production.

Also provided herein is a lyophilized preparation comprising a c-Met antibody drug conjugate prepared by the foregoing method.

In some embodiments, also provided is a lyophilized preparation, wherein the lyophilized preparation can be reconstituted to form any of the foregoing pharmaceutical compositions, preferably reconstituted with water for injection.

Also provided herein is a use of the foregoing pharmaceutical composition or lyophilized preparation in manufacturing a medicament for treating a disease or condition associated with c-Met, wherein the disease or condition is preferably a cancer; more preferably a c-Met-expressing cancer; further more preferably c-Met-expressing gastric cancer, pancreatic cancer, lung cancer (e.g., non-small cell lung cancer), glioblastoma, sarcoma, colorectal cancer, renal cancer, hepatocellular cancer, melanoma or breast cancer; most preferably gastric cancer, pancreatic cancer, non-small cell lung cancer or renal cancer.

Also provided herein is a method of treating and preventing a disease or condition associated with c-Met, comprising administering a therapeutically effective amount of the foregoing pharmaceutical composition or lyophilized preparation to a subject in need thereof, wherein the disease is preferably a cancer; more preferably a c-Met-expressing cancer; even more preferably c-Met-expressing gastric cancer, pancreatic cancer, lung cancer (e.g., non-small cell lung cancer), glioblastoma, sarcoma, colorectal cancer, renal cancer, hepatocellular cancer, melanoma or breast cancer; most preferably gastric cancer, pancreatic cancer, non-small cell lung cancer or renal cancer.

Also provided herein is a product comprising a container, wherein the container comprises the foregoing pharmaceutical composition or lyophilized preparation. The container is preferably a glass bottle, a liquid storage bag or a 316L stainless steel can.

In further embodiments of the present disclosure, the antibody in the c-Met antibody drug conjugate of the pharmaceutical composition comprises a heavy chain sequence having at least 85% (e.g. at least 85%, 86%, 87%, 88%, 89%, 90%, 91%, 92%, 93%, 94%, 95%, 96%, 97%, 98%, or 99%, preferably at least 95%) sequence identity to the amino acid sequence of SEQ ID NO: 24 of WO 2016/165580A1; and a light chain sequence having at least 85% (e.g. at least 85%, 86%, 87%, 88%, 89%, 90%, 91%, 92%, 93%, 94%, 95%, 96%, 97%, 98%, or 99%, preferably at least 95%) sequence identity to the amino acid sequence of SEQ ID NO: 27 of WO 2016/165580A1.

In some embodiments, the preparation is stable at 2-8° C. for at least 3 months, at least 6 months, at least 12 months, at least 18 months, or at least 24 months. In some embodiments, the preparation is stable at 40° C. for at least 7 days, at least 14 days or at least 28 days.

It is appreciated that one, some, or all of the features of the various embodiments described herein can be combined to form other embodiments of the present disclosure. These and other embodiments of the present disclosure will be apparent to those skilled in the art. These and other embodiments of the present disclosure are further described by the following detailed description.

### DETAILED DESCRIPTION OF THE PREFERRED EMBODIMENT

### I. Terms

In order to make the disclosure more readily understood, certain technical and scientific terms are specifically defined below. Unless specifically defined otherwise in this document, all other technical and scientific terms used herein shall be taken to have the same meaning as commonly understood by one of ordinary skill in the art to which this disclosure belongs.

“Buffer” refers to a buffer that is resistant to changes in pH of means of the function of its conjugate acid-base components. The pH value of the buffer of the present disclosure is about 4.5 to 6.0, preferably about 5.0 to 6.0, more preferably about 5.0 to 5.5, most preferably 5.3. Examples of the buffer which controls the pH in such range include acetate buffer, succinate buffer, gluconate buffer, histidine buffer, oxalate buffer, lactate buffer, phosphate buffer, citrate buffer, tartrate buffer, fumarate buffer, glycylglycine buffer and other organic acid buffers. The buffer of the present disclosure is preferably succinate buffer or citrate buffer, more preferably succinate buffer.

“Succinate buffer” refers to a buffer that includes succinate ions. Examples of succinate buffer include succinic acid-sodium succinate, histidine succinate, succinic acid-potassium succinate, succinic acid-calcium succinate, etc. The succinate buffer of the present disclosure is preferably succinic acid-sodium succinate.

“Citrate buffer” refers to a buffer that includes citrate ions. Examples of the citrate buffer include citric acid-sodium citrate, histidine citrate, citric acid-potassium citrate, citric acid-calcium citrate, citric acid-magnesium citrate, etc. The citrate buffer of the present disclosure is citric acid-sodium citrate.

“Pharmaceutical composition” refers to a mixture comprising one or more of the compounds described herein, the physiologically/pharmaceutically acceptable salt thereof or the prodrug thereof, and other chemical component(s). Said other chemical component(s) is, for example, physiological/pharmaceutically acceptable carrier and excipient. The purpose of the pharmaceutical composition is to promote the administration into an organism and facilitate the absorption of the active ingredient, thereby exerting biological activity. As used herein, “pharmaceutical composition” and “preparation” are not mutually exclusive.

The pharmaceutical composition of the present disclosure is capable of achieving a stable effect: the antibody in which can substantially retains its physical stability and/or chemical stability and/or biological activity during storage; preferably, the pharmaceutical composition retains substantially its physical stability, chemical stability and biological activity during storage. The shelf life is generally selected based on the predetermined shelf life of the pharmaceutical composition. There are currently a number of analytical techniques for measuring protein stability, which can measure stability after storage for a selected period of time at a selected temperature.

“Lyophilized preparation” refers to a preparation obtained by lyophilization of a pharmaceutical composition in liquid or solution form, or a liquid or solution preparation under vacuum.

A stable pharmaceutical preparation is one in which no significant change is observed in the following conditions: storage at a refrigerated temperature (2-8° C.) for at least 3 months, preferably 6 months, more preferably 1 year, and even more preferably up to 2 years. In addition, a stable liquid preparation includes such liquid preparation that exhibits the desired characteristic after storage at a temperature including 25° C. and 40° C. for a period including 1 month, 3 months, and 6 months. Typical acceptable criteria for the stability are as follows: typically, no more than about 10%, preferably no more than about 5% of antibody monomer is degraded, as measured by SEC-HPLC. The pharmaceutical liquid preparation is colorless or clear to slightly opalescent white by visual analysis. The concentration, pH and osmolality of the preparation have no more than ±10% of change. No more than 10%, preferably no more than 5% of clipping is observed. No more than 10%, preferably no more than 5% of aggregation is formed.

An antibody “retains its physical stability” in a pharmaceutical preparation if it shows no significant increase of aggregation, precipitation and/or denaturation upon visual examination of color and/or clarity, or as measured by UV light scattering, size exclusion chromatography (SEC) and dynamic light scattering (DLS). The changes of protein conformation can be evaluated by fluorescence spectroscopy (which determines the protein tertiary structure), and by FTIR spectroscopy (which determines the protein secondary structure).

An antibody “retains its chemical stability” in a pharmaceutical preparation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical preparation, if the biological activity of the antibody at a given time is within a predetermined range of the biological activity exhibited at the time the pharmaceutical preparation was prepared. The biological activity of an antibody can be determined, for example, by an antigen binding assay.

“Antibody drug conjugate (ADC)” is formed by attaching a monoclonal antibody or antibody fragment, via a stable chemical linker compound, to a biologically active cytotoxin or a small molecule drug with cell-killing activity, which makes full use of the specificity of the antibody to the specific or high-expression antigen on a tumor cell and the high efficiency of the cytotoxin, thereby avoiding toxic side effects on normal cells. This means that the antibody drug conjugates are capable of precisely binding to tumor cells and reducing the effects on normal cells compared to traditional chemotherapeutic drugs.

“c-Met antibody drug conjugate” refers to an antibody drug conjugate (ADC) formed by the attachment of an antibody targeting c-Met to a cytotoxin or a small molecule drug via a chemical linker. This includes, but is not limited to, ADC-12 of the present disclosure.

The three letter codes and single-letter codes for the amino acid residues used herein are described in J. Biol. Chem. 243, p. 3558 (1968).

The “antibody” used in the present disclosure refers to an immunoglobulin, which is a tetra-peptide chain structure connected together by disulfide bonds between two identical heavy chains and two identical light chains.

In the present disclosure, the light chain of the antibody of the present disclosure can further comprise a light chain constant region including a humanized or murine κ, λ chain or a variant thereof.

In the present disclosure, the heavy chain of the antibody of the present disclosure can further comprise a heavy chain constant region including humanized or murine IgG1, IgG2, IgG3, IgG4 or a variant thereof.

About 110 amino acids sequences adjacent to the N-terminus of the antibody heavy and light chains are highly variable, known as variable region (Fv region); the rest of amino acid sequences close to the C-terminus are relatively stable, known as constant region. The variable region includes three hypervariable regions (HVR) and four relatively conserved framework regions (FR). The three hypervariable regions which determine the specificity of the antibody, are also known as the complementarity determining region (CDR). Each light chain variable region (LCVR) and each heavy chain variable region (HCVR) consists of three CDR regions and four FR regions, with sequential order from the amino terminus to carboxyl terminus in the following order: FR1, CDR1, FR2, CDR2, FR3, CDR3, and FR4. The three CDR regions of the light chain refer to LCDR1, LCDR2, and LCDR3, and the three CDR regions of the heavy chain refer to HCDR1, HCDR2, and HCDR3. The number and position of CDR region amino acid residues in the LCVR and HCVR regions of the antibody or the antigen binding fragments herein comply with known Kabat numbering criteria (LCDR1-3, HCDE2-3), or comply with kabat and chothia numbering criteria (HCDR1).

The antibody of the present disclosure includes a murine antibody, a chimeric antibody and a humanized antibody, preferably a humanized antibody.

The term “murine antibody” in the present disclosure refers to a monoclonal antibody against human PD-L1 prepared according to the knowledge and skills of the field. During the preparation, a test subject is injected with an antigen, and then the hybridoma expressing the antibody having the desired sequence or functional properties is isolated.

The term “chimeric antibody” is an antibody which is formed by fusing the variable region of a murine antibody with the constant region of a human antibody, and the chimeric antibody can alleviate the immune response that is induced by murine antibody. To construct a chimeric antibody, the hybridoma secreting a specific murine monoclonal antibody is first constructed, then a variable region gene is cloned from the mouse hybridoma cells. Subsequently, a constant region gene of a human antibody is cloned as desired, the mouse variable region gene is ligated with the human constant region gene to form a chimeric gene which can be inserted into a human vector, and finally a chimeric antibody molecule is expressed in the eukaryotic or prokaryotic industrial system. In a preferred embodiment of the present disclosure, the light chain of the c-Met chimeric antibody further comprises the light chain constant region of human κ, λ chain, or a variant thereof. The heavy chain of the c-Met chimeric antibody further comprises the heavy chain constant region of human IgG1, IgG2, IgG3, or IgG4, or a variant thereof. The constant region of a human antibody can be selected from the heavy chain constant region of human IgG1, IgG2, IgG3 or IgG4 or a variant thereof, preferably comprising the heavy chain constant region of human IgG2 or IgG4, or IgG4 without ADCC (antibody-dependent cell-mediated cytotoxicity) after amino acid mutation.

The term “humanized antibody”, also known as CDR-grafted antibody, refers to an antibody generated by grafting murine CDR sequences into a variable region framework of a human antibody, namely, an antibody produced from different type of human germline antibody framework sequence. A humanized antibody can overcome the disadvantage of the strong antibody response induced by the chimeric antibody, which carries a large amount of murine protein components. Such framework sequences can be obtained from a public DNA database covering germline antibody gene sequences or published references. For example, germline DNA sequences of human heavy and light chain variable region genes can be found in “VBase” human germline sequence database (available on web www.mrccpe.com.ac.uk/vbase), and Kabat, E A, et al, 1991, Sequences of Proteins of Immunological Interest, 5th Ed. To avoid the decrease in activity while the decrease of immunogenicity, the framework sequences in the variable region of the human antibody are subjected to minimal reverse mutations or back mutations to maintain the activity. The humanized antibody of the present disclosure also comprises a humanized antibody to which CDR affinity maturation is performed by phage display.

The term “homology”, also known as “identity” or “similarity”, refers to the proportion of identical nucleobases or amino acid residues in sequence alignment between the detection sequence and the target sequence.

“Antigen-binding fragment” in the present disclosure refers to a Fab fragment, a Fab′ fragment, or a F(ab′)2 fragment having antigen-binding activity, and a scFv fragment binding to human c-Met, as well as other fragments capable of binding to human c-Met that are formed by utilizing the VH and VL regions of the antibody capable of binding to human c-Met; it comprises one or more CDR regions of antibodies described in the present disclosure, selected from SEQ ID NO: 1 to SEQ ID NO: 2. A Fv fragment comprises a heavy chain variable region and a light chain variable region, without constant region, and it is a minimal antibody fragment possessing all antigen-binding sites. Generally, a Fv antibody further comprises a polypeptide linker between the VH and VL domains, and is capable of forming a structure necessary for antigen binding. Also, different linkers can be used to connect two variable regions of an antibody to form a single polypeptide chain, referred to as a single chain antibody or single chain Fv (scFv). The term “binding to c-Met” in the present disclosure means that it is capable of interacting with human c-Met. The term “antigen-binding sites” in the present disclosure refers to the continuous or discontinuous three-dimensional sites on the antigen, recognized by the antibody or the antigen-binding fragment of the present disclosure.

“c-Met antibody” refers to an antibody capable of specifically binding to c-Met, including, but not limited to, the c-Met antibodies disclosed in WO 2016/165580A1.

“Ab-10” is the c-Met antibody Ab-10 disclosed in WO2016/165580A1, the heavy chain amino acid sequence of which is:

(SEQ ID NO: 24)
QVQLVESGGGVVQPGRSLRLSCAASGFSLSNYGVHWVRQAPGKGLEWLAVI
WSGGSTNYAAAFVSRLTISKDNSKNTVYLQMNSLRAEDTAVYYCARNHDNP
YNYAMDYWGQGTTVTVSSASTKGPSVFPLAPCSRSTSESTAALGCLVKDYF
PEPVTVSWNSGALTSGVHTFPAVLQSSGLYSLSSVVTVPSSNFGTQTYTCN
VDHKPSNTKVDKTVERKCCVECPPCPAPPVAGPSVFLFPPKPKDTLMISRT
PEVTCVVVDVSHEDPEVQFNWYVDGVEVHNAKTKPREEQFNSTFRVVSVLT
VVHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYSK
LTVDKSRWQQGNVFSCSVMHEALHNHYTQKSLSLSPGK.

The light chain amino acid sequence is

(SEQ ID NO: 27)
DIVLTQSPDSLAVSLGERATINCRADKSVSTSTYNYLHWYQQKPGQPPKLL
IYLASNLASGVPDRFSGSGSGTDFTLTISSLQAEDVAVYYCQHSRDLPPTF
GQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC.

“ADC-12” is a c-Met antibody drug conjugate formed by the attachment of Ab-10 to a small molecule toxin via a chemical linker, having a structure represented by ADC-12 as follows:

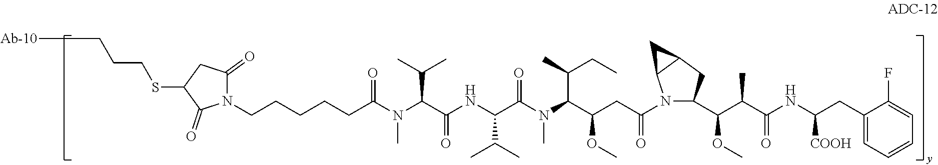

wherein, y ranges from 1 to 8, preferably from 2 to 5; y can be a decimal.

Methods for producing and purifying antibodies and antigen-binding fragments are well known in the art and can be found, for example, in Antibody Experimental Technology Guide of Cold Spring Harbor, Chapters 5-8 and 15. The antibody or the antigen-binding fragments of the present disclosure is genetically engineered to introduce one or more human framework regions (FRs) to a non-human derived CDR region. Human FR germline sequences can be obtained from ImMunoGeneTics (IMGT) and MOE software, via the website http://imgt.cines.fr, or from The Immunoglobulin FactsBook, 2001ISBN012441351.

The engineered antibody or antigen-binding fragments of the present disclosure can be prepared and purified by conventional methods. For example, cDNA sequences encoding a heavy chain and a light chain can be cloned and recombined into a GS expression vector. The recombined immunoglobulin expression vector can then be stably transfected into CHO cells. As a more recommended method well known in the art, mammalian expression systems will result in glycosylation of antibodies, typically at the highly conserved N-terminus in the Fc region. Stable clones can be obtained through expression of an antibody specifically binding to human c-Met. Positive clones can be expanded in serum-free culture medium for antibody production in bioreactors. Culture medium, into which an antibody has been secreted, can be purified by conventional techniques. For example, the medium can be conveniently applied by a Protein A or G Sepharose FF column that has been equilibrated with adjusted buffer. The column is washed to remove nonspecific binding components. The bound antibody is eluted by pH gradient and antibody fragments are detected by SDS-PAGE, and then pooled. The antibody can be filtered and concentrated using common techniques. Soluble aggregate and multimers can be effectively removed by common techniques, including size exclusion or ion exchange. The obtained product can be immediately cryopreserved, for example at −70° C., or can be lyophilized.

“Conservative modifications” or “conservative replacement or substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those skilled in the art recognize that, in general, single amino acid substitutions in non-essential regions of a polypeptide does not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to disrupt biological activity.

“Identity” refers to sequence similarity between two proteins or between two polypeptides. When a position in both of the two compared sequences is occupied by the same amino acid monomer subunit, e.g., if a position in both of two polypeptides is occupied by the same amino acid monomer subunit, then the molecules are identical at that position. Examples of algorithms suitable for determining the percent of sequence identity and similarity are the BLAST and BLAST 2.0 algorithms, which are described in Altschul et al. (1990) J Mol. Biol. 215: 403-410 and Altschul et al. (1977) Nucleic Acids Res. 25:3389-3402, respectively. Software for performing BLAST analyses is publicly available at the National Center for Biotechnology Information (http://www.ncbi.nlm.nih.gov/).

“Administration” and “treatment”, when applying to an animal, human, experimental subject, cell, tissue, organ, or biological fluid, refer to contacting an exogenous pharmaceutical, therapeutic, diagnostic agent, or composition with the animal, human, subject, cell, tissue, organ, or biological fluid. “Administration” and “treatment” can refer, e.g., to therapeutic, pharmacokinetic, diagnostic, research, and experimental methods. Treatment of a cell encompasses contacting a reagent with the cell, as well as contacting a reagent with a fluid, where the fluid is in contact with the cell. “Administration” and “treatment” also means in vitro and ex vivo treatments, e.g., of a cell, by a reagent, diagnostic, binding compound, or by another cell. “Treatment”, as it applies to a human, veterinary, or a research subject, refers to therapeutic treatment, prophylactic or preventative measures, research and diagnostic applications.

“Treat” means to administer a therapeutic agent, such as a composition comprising any of the binding compounds of the present disclosure, internally or externally to a patient having one or more disease symptoms for which the agent has known therapeutic activity. Typically, the agent is administered in an amount effective to alleviate one or more disease symptoms in the treated patient or population, so as to induce the regression of or inhibit the progression of such symptom(s) to any clinically measurable degree. The amount of a therapeutic agent that is effective to alleviate any particular disease symptom (also referred to “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present disclosure (e.g., a treatment method or article of manufacture) may not be effective in alleviating the disease symptom(s) of interest in every patient, it should alleviate the target disease symptom(s) of interest in a statistically significant number of patients as determined by any statistical test known in the art such as the Student's t-test, the chi-square test, the U-test according to Mann and Whitney, the Kruskal-Wallis test (H-test), Jonckheere-Terpstra-test and the Wilcoxon-test.

“Effective amount” encompasses an amount sufficient to ameliorate or prevent a symptom or sign of a medical condition. Effective amount also means an amount sufficient to allow or facilitate diagnosis. An effective amount for a particular patient or veterinary subject can vary depending on factors such as the condition being treated, the general health of the patient, the route and dose of administration and the severity of side effects. An effective amount can be the maximal dose or dosing protocol that avoids significant side effects or toxic effects.

“Tm value” refers to the thermal denaturation midpoint of the protein, namely, the temperature at which half of the protein is unfolded and the spatial structure of the protein is destroyed. Therefore, the higher the Tm value is, the higher the thermal stability of the protein will be.

### II. Embodiments and Test Embodiments

The present disclosure is further described with reference to the following embodiments, which are not intended to limit the scope of the disclosure. The experimental methods in the embodiments of the present disclosure which do not specify the specific conditions are usually carried out according to conventional conditions or according to the conditions recommended by the manufacturer of the raw material or the commodity. Reagents without indicating specific source are routine reagents commercially available.

### Embodiment 1: Preparation of ADC-12 by Coupling Anti-c-Met Antibody Ab-10 with Toxin

### 1. Preparation of the Toxin

### (S)-2-((2R,3R)-3-((1S,3S,5S)-2-((3R,4S,5S)-4-((S)—N,3-Dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propionic Acid

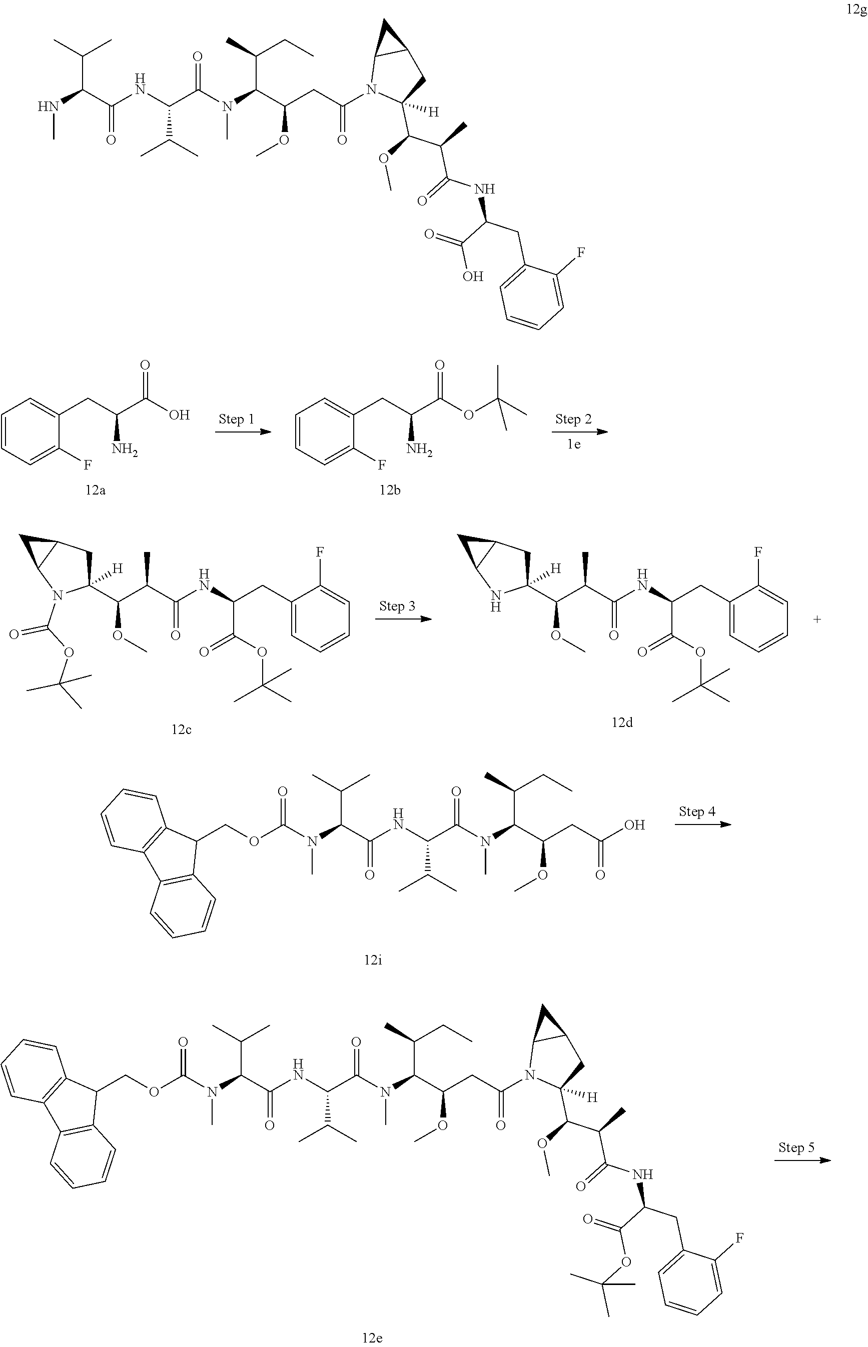

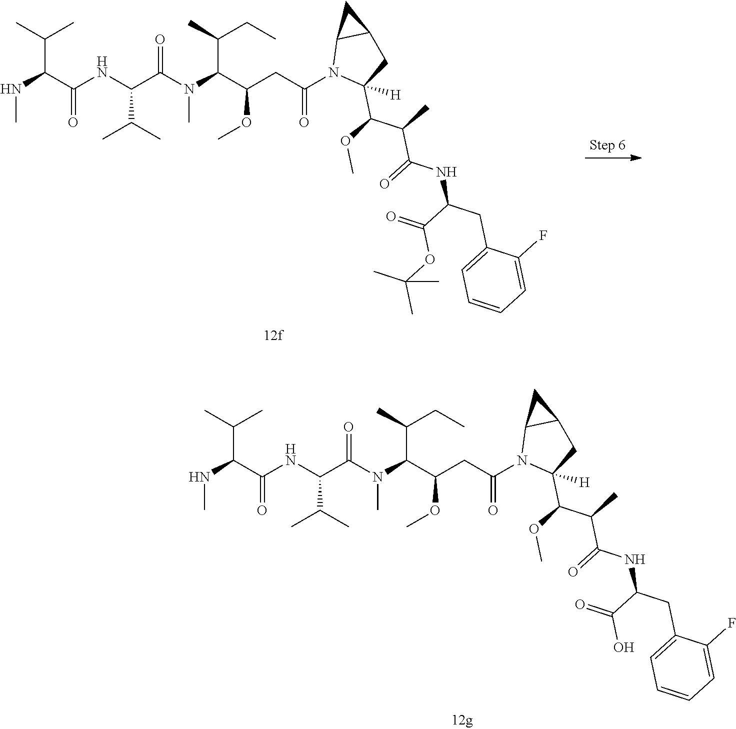 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C]

### Step 1

### Tert-butyl (S)-2-amino-3-(2-fluorophenyl)propanoate

The raw material (S)-2-amino-3-(2-fluorophenyl)propionic acid 12a (400 mg, 2.18 mmol, Shanghai HC Biotech Co., Ltd., CAT # F2202) was dissolved in 10 mL of tert-butyl acetate. Perchloric acid (300 mg (70%), 3.3 mmol) was added and the mixture was stirred at room temperature for 16 hours. After completion of the reaction, 6 mL of water were added, followed by liquid separation. The organic phase was washed with saturated sodium bicarbonate aqueous solution (5 mL). The aqueous phase was adjusted to pH=8 with saturated sodium bicarbonate aqueous solution and extracted with dichloromethane (5 mL×3). The organic phases were combined, washed with water (3 mL) and saturated sodium chloride aqueous solution (5 mL) successively, and dried over anhydrous sodium sulfate, followed by filtration. The filtrate was concentrated under reduced pressure to give a crude product of the title compound: tert-butyl (S)-2-amino-3-(2-fluorophenyl)propanoate 12b (390 mg, yellow oil). The crude product was directly used in the next step without purification.

### Step 2

### Tert-butyl (1S,3S,5S)-3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexan-2-carboxylate

The raw material (2R,3R)-3-((1S,3S,5S)-2-(tert-butoxycarbonyl)-2-azabicyclo[3.1.0]hexan-3-yl)-3-methoxy-2-methylpropionic acid 1e (100 mg, 0.334 mmol) was dissolved in 6 mL of mixed solvent of dichloromethane and dimethylformamide (V/V=5:1), followed by addition of the crude product of tert-butyl (S)-2-amino-3-(2-fluorophenyl)propanoate 12b (80 mg, 0.334 mmol), N,N-diisopropylethylamine (0.29 mL, 1.67 mmol) and 2-(7-azabenzotriazol-1-yl)-N,N,N′,N′-tetramethyluronium hexafluorophosphate (152.3 mg, 0.40 mmol). The reaction system was stirred at room temperature for 1 hour under argon atmosphere. After completion of the reaction, 10 mL of water were added, followed by liquid separation. The dichloromethane phase was washed with saturated sodium chloride aqueous solution (10 mL) and dried over anhydrous sodium sulfate, followed by filtration. The filtrate was concentrated under reduced pressure. The obtained residue was purified by silica gel column chromatography with eluent system B to give the product of the title compound tert-butyl (1S,3S,5S)-3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexan-2-carboxylate 12c (173 mg, colorless liquid) with a yield of 99.5%.

MS m/z (ESI): 521.2 [M+1]

### Step 3

### Tert-butyl (S)-2-((2R,3R)-3-((1S,3S,5S)-2-azabicyclo[3.1.0]hexan-3-yl)-3-methoxy-2-methylpropanamido)-3-(2-fluorophenyl)propanoate

The raw material tert-butyl (1S,3S,5S)-3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexan-2-carboxylate 12c (173 mg, 0.33 mmol) was dissolved in 2 mL of dioxane, followed by addition of 5.6 M solution of hydrogen chloride in dioxane (0.21 mL, 1.16 mmol). The mixture was stirred at room temperature for 1 hour under argon atmosphere, and placed in a refrigerator at 0° C. for 12 hours. After completion of the reaction, the reaction solution was concentrated under reduced pressure, followed by dilution with 5 mL of dichloromethane and addition of 10 mL of saturated sodium bicarbonate aqueous solution. The mixture was stirred for 10 minutes, followed by liquid separation. The aqueous phase was extracted with dichloromethane (5 mL×3). The dichloromethane phases were combined, washed with saturated sodium chloride aqueous solution (10 mL) and dried over anhydrous sodium sulfate, followed by filtration. The filtrate was concentrated under reduced pressure to give a crude product of the title compound tert-butyl (S)-2-((2R,3R)-3-((1S,3S,5S)-2-azabicyclo[3.1.0]hexan-3-yl)-3-methoxy-2-methylpropanamido)-3-(2-fluorophenyl)propanoate 12d (77 mg, yellow liquid). The crude product was directly used in the next step without purification.

MS m/z (ESI): 421.2 [M+1]

### Step 4

### Tert-butyl (S)-2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate

The crude product of tert-butyl (S)-2-((2R,3R)-3-((1S,3S,5S)-2-azabicyclo[3.1.0]hexan-3-yl)-3-methoxy-2-methylpropanamido)-3-(2-fluorophenyl)propanoate 12d (77 mg, 0.183 mmol) and (5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecane-14-carboxylic acid 12i (116.8 mg, 0.183 mmol, prepared by the method disclosed in WO2013072813, see the synthesis procedure of compound #8 on pages 115-119 of the specification) were dissolved in 6 mL of mixed solvent of dichloromethane and dimethylformamide (V/V=5:1), followed by addition of N,N-diisopropylethylamine (0.16 mL, 0.915 mmol) and 2-(7-azabenzotriazol-1-yl)-N,N,N′,N′-tetramethyluronium hexafluorophosphate (84 mg, 0.22 mmol). The reaction system was stirred at room temperature for 1 hour under argon atmosphere. After completion of the reaction, 10 mL of water were added, followed by liquid separation. The dichloromethane phase was washed with saturated sodium chloride aqueous solution (10 mL) and dried over anhydrous sodium sulfate, followed by filtration. The filtrate was concentrated under reduced pressure. The obtained residue was purified by silica gel column chromatography with eluent system B to give the product of title compound tert-butyl (S)-2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate 12e (190.5 mg, yellow and sticky) with a yield of 100%.

MS m/z (ESI): 1040.6 [M+1]

### Step 5

### Tert-butyl (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate

The raw material tert-butyl (S)-2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate 12e (190.5 mg, 0.183 mmol) was dissolved in 1.5 mL of dichloromethane, followed by addition of 2 mL of diethylamine. The reaction system was stirred at room temperature for 3 hours under argon atmosphere. After completion of the reaction, the reaction mixture was concentrated under reduced pressure to give a crude product of the title compound tert-butyl (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate 12f (150 mg, yellow sticky substance). The crude product was directly used in the next step without purification.

MS m/z (ESI): 818.5 [M+1]

### Step 6

### (S)-2-((2R,3R)-3-((1S,3S,5S)-2-((3R,4S,5S)-4-((S)—N,3-Dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic Acid

The crude product of tert-butyl (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate 12f (150 mg, 0.183 mmol) was dissolved in 1 mL of dioxane, followed by addition of 3 mL of 5.6 M solution of hydrogen chloride in dioxane. The reaction mixture was stirred at room temperature for 12 hours under argon atmosphere. After completion of the reaction, the reaction mixture was concentrated under reduced pressure, and the residual solvent was removed by evaporation with diethyl ether. The obtained residue was purified by high performance liquid chromatography to give the product of the title compound (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid 12g (28 mg, white powder solid) with a yield of 20%.

MS m/z (ESI): 762.7 [M+1]

¹H NMR (400 MHz, CD₃OD): δ 7.38-7.18 (m, 2H), 7.13-7.01 (m, 2H), 4.80-4.67 (m, 2H), 4.30-4.15 (m, 1H), 4.13-4.01 (m, 1H), 3.96-3.83 (m, 2H), 3.75-3.60 (m, 2H), 3.42-3.11 (m, 9H), 3.06-2.95 (m, 1H), 2.70-2.58 (m, 4H), 2.28-2.01 (m, 4H), 1.88-1.70 (m, 3H), 1.57-1.25 (m, 4H), 1.22-0.95 (m, 18H), 0.92-0.80 (m, 4H), 0.78-0.65 (m, 1H).

### 2. Preparation of Toxin Intermediate

### (S)-2-((2R,3R)-3-((1S,3S,5S)-2-((3R,4S,5S)-4-((S)-2-((S)-2-(6-(2,5-Dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-(2-fluorophenyl)propanoic Acid

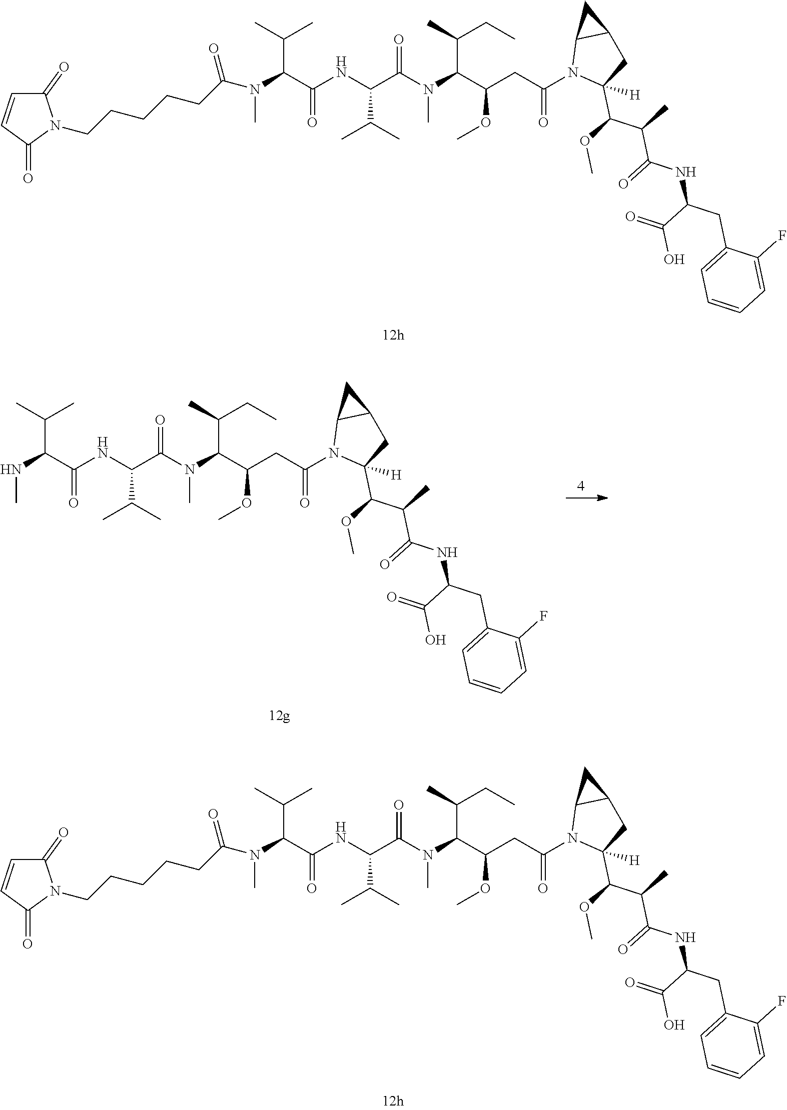 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C]

The raw material (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid 12g (25 mg, 0.033 mmol) was dissolved in 3 mL of dichloromethane, followed by addition of N,N-diisopropylethylamine (0.029 mL, 0.164 mmol). A pre-prepared solution of 6-(2,5-dioxo-2,5-dihydro-1H-pyrrol-1-yl)hexanoyl chloride 4 (11.3 mg, 0.049 mmol) in dichloromethane was added to the reaction system under argon atmosphere and the mixture was stirred at room temperature for 3 hours. After completion of the reaction, 5 mL of water were added, and the mixture was stirred for 20 minutes, followed by liquid separation. The organic phase was dried over anhydrous sodium sulfate, followed by filtration. The filtrate was concentrated under reduced pressure and the residue was purified by high performance liquid chromatography to give the product of the title compound (S)-2-((2R,3R)-3-41S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-(2-fluorophenyl)propanoic acid 12h (7 mg, yellow and sticky) with a yield of 22.4%.

MS m/z (ESI): 955.4 [M+1]

¹H NMR (400 MHz, CD₃OD): δ 7.36-7.30 (m, 1H), 7.29-7.21 (m, 1H), 7.17-7.02 (m, 2H), 6.83-6.79 (m, 2H), 4.81-4.71 (m, 2H), 4.69-4.55 (m, 2H), 4.25-4.15 (m, 1H), 4.13-4.04 (m, 1H), 3.96-3.85 (m, 2H), 3.70-3.61 (m, 1H), 3.55-3.46 (m, 3H), 3.40-3.21 (m, 4H), 3.18-3.10 (m, 2H), 3.07-2.96 (m, 4H), 2.67-2.56 (m, 2H), 2.54-2.34 (m, 3H), 2.29-2.17 (m, 2H), 2.10-1.99 (m, 1H), 1.89-1.57 (m, 7H), 1.52-1.28 (m, 6H), 1.21-1.11 (m, 4H), 1.07-0.96 (m, 6H), 0.95-0.81 (m, 12H), 0.80-0.69 (m, 1H).

### 3. Preparation of Antibody Toxin Conjugate

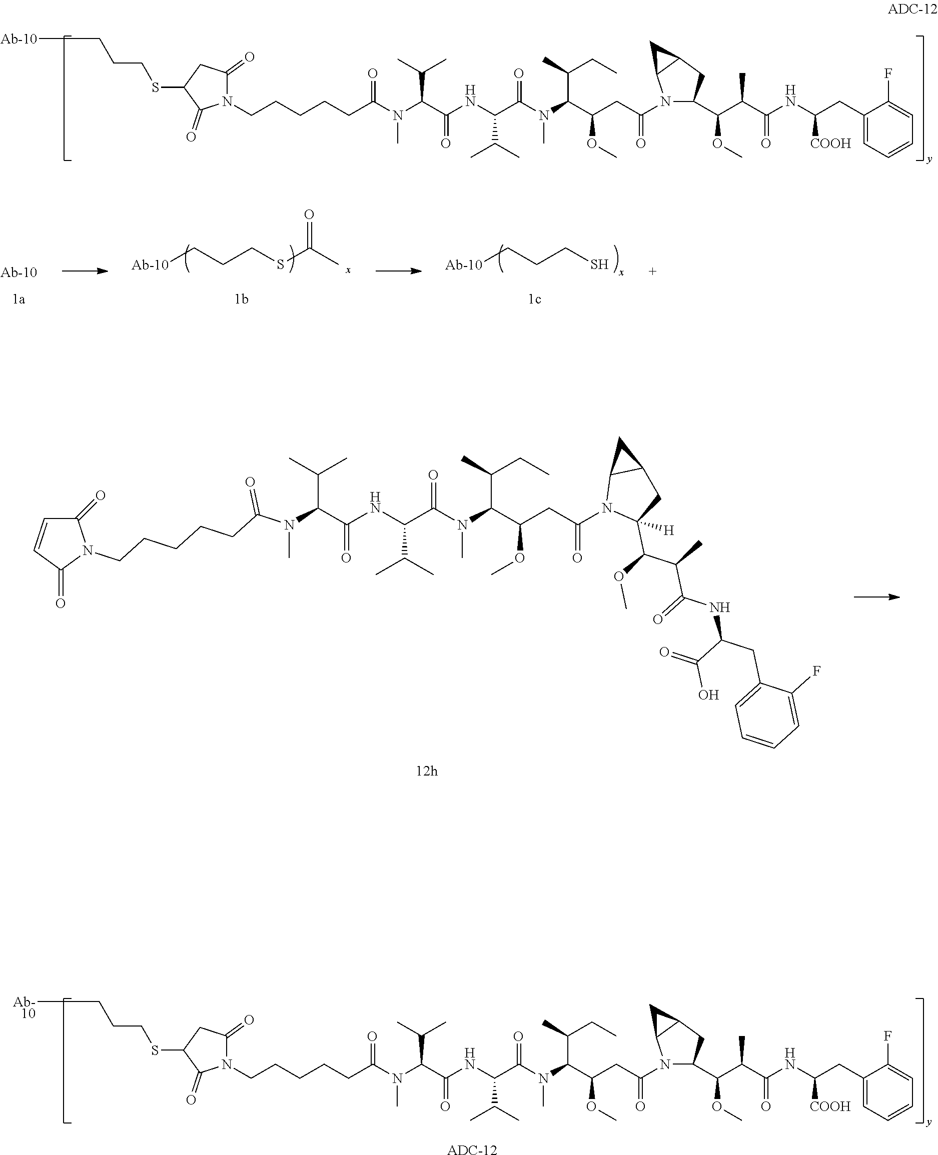

Compound 12h (1.2 mg, 1.2 μmol) was dissolved in 0.3 mL of acetonitrile, followed by addition of Ab-10 monoclonal antibody-propanol 1c solution (6.17 mg/mL, 3.0 mL). The reaction was carried out at 25° C. for 4 hours while shaking, followed by desalting purification on a Sephadex G25 gel column (elution phase: 0.05 M PBS solution, pH 6.5), and filtration through a 0.2 μm filter under sterile conditions to give a solution of the title product ADC-12 in PBS buffer (3.3 mg/mL, 5.0 mL), which was stored at 4° C.

The preparation method of Ab-10 monoclonal antibody was the same as the preparation method of Ab-10 monoclonal antibody disclosed in Embodiments 1-3 and 5-6 of WO2016/165580A1.

Q-TOF LC/MS: Characteristic peaks: 148119.6 (M_{Ab}+OD), 149150.5 (M_{Ab}+1D), 150221.1 (M_{Ab}+2D), 151265.1 (M_{Ab}+3D), 152314.3 (M_{Ab}+4D).

Average value: y=1.6.

The Preparation Process of Stabilizing Preparations of ADC is as Follows:

Step 1: The ADC-12 stock solution was filtered and tested for sterility in central control. The stock solution was passed through a 0.22 μm PVDF filter and the filtrate was collected. The ADC-12 is an anti-c-Met antibody ADC, wherein the c-Met antibody is Ab-10, having a heavy chain as shown in SEQ ID NO: 24 and a light chain as shown in SEQ ID NO: 27 in WO2016/165580A1.

Step 2: The loading was adjusted to 4.2 mL. The filtrate was filled into a 15 mL vial, and half-sealed with a stopper. A sample was taken for measuring the uniformity of loading in central control at the beginning, middle and end of filling.

Step 3: The filled and sealed liquid medicine was placed in a lyophilization chamber to perform the lyophilization process. The lyophilization included sequential steps of pre-freezing, primary drying and secondary drying. After completion of lyophilization, the vial was stoppered under vacuum. Exemplary lyophilization parameters were as follows:

| | | | | Vacuum |
|---|---|---|---|---|
| | Temperature | Set time | Hold time | degree |
| Parameters | (° C.) | (min) | (min) | (mbar) |
| Pre-freezing | −5 | 10 | 60 | / |
| | −45 | 40 | 180 | / |
| Primary drying | −20 | 100 | 2160 | 0.1 |
| Secondary | 25 | 60 | 450 | 0.01 |
| drying | | | | |

Step 4: The capping machine was run, adding an aluminum cap and carrying out the capping.

Step 5: Visual inspection was used to confirm that the product had no defects such as collapse and inaccurate loading. The vial labels were printed and pasted; and the box labels were printed, followed by folding the tray, boxing and sticking the box labels.

The experiment was designed based on the buffer system, buffer concentration, pH value, saccharide type and saccharide concentration of ADC-12 preparations. The Tm value of the sample was determined by DSC technique, and the prescription of the preparation was initially screened.

The test was designed with the buffer system, buffer concentration, pH value, saccharide type and saccharide concentration as the factors and the Tm value as the response value, generating the designed tables. The experiments were performed according to the experimental groups of the designed tables, and the Tm value was determined.

In the embodiments, Tofflon LYO-3 (SIP, CIP) vacuum lyophilization machine was used to perform the lyophilization. Agilent 1200 DAD high pressure liquid chromatograph (Waters Xbridge Protein BEH SEC 200A column) was used to measure SE-HPLC. Beckman PA800 plus capillary electrophoresis apparatus (SDS-Gel MW Analysis Kit) was used to measure CE-SDS. GE MicroCal VP-Capillary DSC differential scanning calorimeter was used to measure the protein heat denaturation temperature (Tm). Malvern Zetasizer Nano ZS nanoparticle size potentiometer was used to measure DLS (Dynamic Light Scattering) average particle size.

### Embodiment 2

The preparations of ADC-12 at a concentration of 1 mg/mL were prepared in the following buffers:

1) 20 mM acetic acid (sodium acetate), pH 5.0

2) 20 mM acetic acid (sodium acetate), pH 5.5

3) 20 mM succinic acid (sodium succinate), pH 5.5

4) 20 mM succinic acid (sodium succinate), pH 6.0

5) 20 mM citric acid (sodium citrate), pH 5.0

6) 20 mM citric acid (sodium citrate), pH 5.5

7) 20 mM citric acid (sodium citrate), pH 6.0

8) 20 mM histidine (hydrochloric acid), pH 5.5

9) 20 mM histidine (hydrochloric acid), pH 6.0

10) 20 mM disodium hydrogen phosphate (sodium dihydrogen phosphate), pH 6.0

The thermal stability of ADC-12 in each preparation was measured by differential scanning calorimetry (DSC) (see Table 1 for the test results).

TABLE 1
DSC results of screening ADC-12 buffer system-pH

| | α,α-trehalose | | | | |
|---|---|---|---|---|---|
| ADC-12 | dihydrate | | | Tmₒₙₛₑₜ | Tm |
| (mg/mL) | (mg/mL) | pH | Buffer system | (° C.) | (° C.) |
| 1 | N/A | 5.0 | 20 mM acetic acid | 56.76 | 78.01 |
| | | | (sodium acetate) | | |
| | | 5.5 | 20 mM acetic acid | 59.29 | 79.28 |
| | | | (sodium acetate) | | |
| | | 5.5 | 20 mM succinic acid | 58.49 | 78.65 |
| | | | (sodium succinate) | | |
| | | 6.0 | 20 mM succinic acid | 60.46 | 79.55 |
| | | | (sodium succinate) | | |
| | | 5.0 | 20 mM citric acid | 51.49 | 76.06 |
| | | | (sodium citrate) | | |
| | | 5.5 | 20 mM citric acid | 58.19 | 78.43 |
| | | | (sodium citrate) | | |
| | | 6.0 | 20 mM citric acid | 60.92 | 79.67 |
| | | | (sodium citrate) | | |
| | | 5.5 | 20 mM histidine | 54.08 | 76.42 |
| | | | (hydrochloric acid) | | |
| | | 6.0 | 20 mM histidine | 58.8 | 78.47 |
| | | | (hydrochloric acid) | | |
| | | 6.0 | 20 mM sodium | 61.37 | 79.55 |
| | | | dihydrogen | | |
| | | | phosphate (disodium | | |
| | | | hydrogen phosphate) | | |

Note:
N/A means that the ingredient was not added.

The results indicate that the histidine (hydrochloric acid) buffer system is significantly lower than other groups. The acetate buffer system may cause a pH shift due to the volatilization during lyophilization. The buffer range of the phosphate buffer (pH 6.0-8.0) is unduly overlapping with the isoelectric point range of ADC-12 and should not be used. Therefore, two types of buffer systems: succinate and citrate with relatively high Tmₒₙₛₑₜ and Tₘ are selected.

ADC-12 preparation was prepared with 10 mM succinic acid (sodium succinate) or citric acid (sodium citrate) at pH 5.5 as buffer, containing 60 mg/mL sucrose, 0.2 mg/mL polysorbate 20 and 20 mg/mL ADC-12. It was filled into a 15 mL vial with 4 mL/vial, lyophilized and sealed with a rubber stopper for lyophilization. The lyophilized products were placed at 25° C. for testing. The results indicate that the succinic acid (sodium succinate) system is slightly better than the citric acid (sodium citrate) system.

TABLE 2
Stability results of ADC-12 in different buffer systems at 25° C.

| | | | SEC (%) | | Non-reduced | |
|---|---|---|---|---|---|---|
| Buffer system | Time | | Monomer | Polymer | CE-SDS (%) | Appearance |
| Succinic acid | M | 0 | 96.4 | 3.6 | 93.4 | Clear and transparent, |
| (sodium succinate) | | | | | | light blue opalescence |
| | D | 15 | 97.0 | 3.0 | 93.4 | Clear and transparent, |
| | | | | | | light blue opalescence |
| | M | 1 | 97.0 | 3.0 | 94.1 | Clear and transparent, |
| | | | | | | light blue opalescence |
| | M | 3 | 96.1 | 3.9 | 93.5 | Clear and transparent, |
| | | | | | | light blue opalescence |
| | M | 6 | 95.9 | 4.1 | 91.1 | Clear and transparent, |
| | | | | | | light blue opalescence |
| Citric acid | M | 0 | 95.8 | 4.2 | 93.7 | Clear and transparent, |
| (Sodium citrate) | | | | | | light blue opalescence |
| | D | 15 | 96.0 | 4.0 | 93.4 | Clear and transparent, |
| | | | | | | light blue opalescence |
| | M | 1 | 96.8 | 3.2 | 93.9 | Clear and transparent, |
| | | | | | | light blue opalescence |
| | M | 3 | 95.9 | 4.1 | 93.7 | Clear and transparent, |
| | | | | | | light blue opalescence |
| | M | 6 | 95.7 | 4.1 | 89.5 | Clear and transparent, |
| | | | | | | light blue opalescence |

Note:
M 0 refers to the 0 month; D 15 refers to the 15^{th} day; M 3 refers to the 3^{rd} month; and M 6 refers to the 6^{th} month.

### Embodiment 3

The ADC-12 preparations were prepared with a buffer containing 10 mM succinic acid-sodium succinate at pH 4.8-5.8, containing 60 mg/mL α,α-trehalose dihydrate, 0.2 mg/mL polysorbate 20 and 20 mg/mL ADC-12. Each preparation was filtered, filled into a 15 mL neutral borosilicate glass controlled injection bottle with 4 mL/bottle, lyophilized and sealed with a rubber stopper. The lyophilized products were stored at 25° C. for stability analysis. The stability results of ADC-12 at different pH at 25° C. for 0-6 months were shown in Table 3. The results indicate that ADC-12 is quite stable at pH 5.0-5.5.

TABLE 3
Stability results of ADC-12 at different pH at 25° C.

| | | | SEC (%) | | Non-reduced |
|---|---|---|---|---|---|
| pH | Time | Appearance | Monomer | Polymer | CE-SDS |
| 4.8 | M 0 | Low clarity, opalescence, white | 96.80 | 3.20 | 91.21 |
| | | insoluble matters | | | |
| | M 3 | Clear and transparent, blue | 95.86 | 4.14 | 92.55 |
| | | opalescence | | | |
| | M 6 | Clear and transparent, blue | 96.74 | 3.26 | 90.48 |
| | | opalescence | | | |
| 5.0 | M 0 | Low clarity, with opalescence | 96.30 | 3.70 | 91.28 |
| | M 3 | Clear and transparent, blue | 95.28 | 4.46 | 93.53 |
| | | opalescence | | | |
| | M 6 | Clear and transparent, blue | 96.21 | 3.79 | 90.54 |
| | | opalescence | | | |
| 5.3 | M 0 | Relatively clear | 96.50 | 3.50 | 91.39 |
| | M 3 | Clear and transparent, blue | 95.42 | 4.55 | 92.51 |
| | | opalescence | | | |
| | M 6 | Clear and transparent, blue | 96.37 | 3.63 | 90.68 |
| | | opalescence | | | |
| 5.5 | M 0 | Relatively clear | 96.50 | 3.50 | 91.44 |
| | M 3 | Clear and transparent, blue | 95.04 | 4.90 | 92.57 |
| | | opalescence | | | |
| | M 6 | Clear and transparent, blue | 95.87 | 4.13 | 89.83 |
| | | opalescence | | | |
| 5.8 | M 0 | Relatively clear | 95.80 | 4.20 | 90.45 |
| | M 3 | Clear and transparent, blue | 94.25 | 5.57 | 92.23 |
| | | opalescence | | | |
| | M 6 | Clear and transparent, blue | 95.00 | 5.00 | 89.26 |
| | | opalescence | | | |

Note:
M 0 refers to the 0 month; M 3 refers to the 3^{rd} month; and M 6 refers to the 6^{th} month.

### Embodiment 4

ADC-12 preparations were prepared with 60 mg/mL α,α-trehalose dihydrate or sucrose at pH 5.5 as buffer, containing 10 mM succinic acid-sodium succinate, 0.2 mg/mL polysorbate 20 and 20 mg/mL ADC-12. Each preparation was filtered and filled into a 15 mL vial with 4 mL/vial, lyophilized and sealed with a rubber stopper for lyophilization. The lyophilized products were stored at 25° C. and 2-8° C. for stability analysis. The results indicate that ADC-12 is more stable in the trehalose system.

TABLE 4
Stability results of ADC-12 lyophilized product at 25° C. when screening saccharide

| | | | SEC | | Non-reduced |
|---|---|---|---|---|---|
| Saccharide | Time | Appearance | Monomer % | Polymer % | CE-SDS |
| Trehalose | M 0 | Relatively clear | 96.50% | 3.50% | 91.44% |
| | M 3 | Clear and transparent, blue | 94.35% | 5.57% | 92.65% |
| | | opalescence | | | |
| | M 6 | Clear and transparent, blue | 95.87% | 4.13% | 89.83% |
| | | opalescence | | | |
| Sucrose | M 0 | Obvious white opalescence, | 95.70% | 4.30% | 90.35% |
| | | low clarity | | | |
| | M 3 | Clear, white opalescence | 93.95% | 5.94% | 92.28% |
| | M 6 | Clear, white opalescence | 95.18% | 4.82% | 90.80% |

Note:
M 0 refers to the 0 month; M 3 refers to the 3^{rd} month; and M 6 refers to the 6^{th} month.

TABLE 5
Stability results of ADC-12 lyophilized product
at 2-8° C. when screening saccharide

| | | | SEC | | Non-reduced |
|---|---|---|---|---|---|
| Saccharide | Time | Appearance | Monomer % | Polymer % | CE-SDS |
| Trehalose | M 0 | Relatively clear | 96.50% | 3.50% | 91.44% |
| | M 3 | Clear and transparent, blue | 95.04% | 4.90% | 92.57% |
| | | opalescence | | | |
| | M 6 | Clear and transparent, blue | 96.34% | 3.66% | 91.20% |
| | | opalescence | | | |
| Sucrose | M 0 | Obvious white opalescence, | 95.70% | 4.30% | 90.35% |
| | | low clarity | | | |
| | M 3 | Clear, white opalescence | 94.46% | 5.46% | 92.69% |
| | M 6 | Clear, white opalescence | 95.66% | 4.34% | 90.23% |

Note:
M 0 refers to the 0 month; M 3 refers to the 3^{rd} month; and M 6 refers to the 6^{th} month.

### Embodiment 5

The ADC-12 preparations containing 10 mM succinic acid-sodium succinate, 60 mg/mL α,α-trehalose dihydrate, and 20 mg/mL ADC-12, were prepared in a buffer of pH 5.5 containing the following surfactants at different concentrations:

1) Without surfactant

2) 0.05 mg/mL polysorbate 20

3) 0.1 mg/mL polysorbate 20

4) 0.2 mg/mL polysorbate 20

5) 0.4 mg/mL polysorbate 20

After completion of the sample preparation, the sample was placed in a −35° C. refrigerator for 12 hours, and then transferred to 2-8° C. for 12 hours, being one freeze-thaw cycle. A total of 5 cycles were repeated. The stability results indicate that 0.05-0.4 mg/mL polysorbate 20 effectively prevent the aggregation of ADC-12 during the freeze-thaw process.

TABLE 6

| Concentration of | | | | | |
|---|---|---|---|---|---|
| polysorbate 20 | | SEC | | Non-reduced | |
| (mg/mL) | Time | Monomer % | Polymer % | CE-SDS % | Appearance |
| 0 | 0 | 97.15 | 2.85 | 92.12 | With large amounts of |
| | | | | | visible foreign matter |
| | Cycle 1 | 96.94 | 3.06 | 91.25 | With large amounts of |
| | | | | | visible foreign matter |
| | Cycle 2 | 96.85 | 3.15 | 91.61 | With large amounts of |
| | | | | | visible foreign matter |
| | Cycle 3 | 96.90 | 3.1 | 91.57 | With large amounts of |
| | | | | | visible foreign matter |
| | Cycle 5 | 96.32 | 3.68 | 90.99 | With large amounts of |
| | | | | | visible foreign matter |
| 0.05 | 0 | 96.76 | 3.24 | 91.80 | Clear and transparent |
| | Cycle 1 | 96.80 | 3.2 | 92.71 | With fine particles |
| | Cycle 2 | 96.56 | 3.44 | 92.01 | With fine particles |
| | Cycle 3 | 96.50 | 3.5 | 91.63 | With fine particles |
| | Cycle 5 | 95.99 | 4.01 | 90.77 | With fine particles |
| 0.1 | 0 | 96.76 | 3.24 | 91.34 | Clear and transparent |
| | Cycle 1 | 96.77 | 3.23 | 92.54 | With fine particles |
| | Cycle 2 | 96.54 | 3.46 | 91.77 | With fine particles |
| | Cycle 3 | 96.46 | 3.54 | 91.10 | With fine particles |
| | Cycle 5 | 95.95 | 4.05 | 90.99 | With fine particles |
| 0.2 | 0 | 97.07 | 2.93 | 92.02 | Clear and transparent |
| | Cycle 1 | 97.00 | 3 | 91.97 | With fine particles |
| | Cycle 2 | 96.82 | 3.18 | 91.79 | With fine particles |
| | Cycle 3 | 96.81 | 3.19 | 91.67 | With fine particles |
| | Cycle 5 | 96.26 | 3.74 | 91.11 | With fine particles |
| 0.4 | 0 | 96.91 | 3.09 | 91.37 | Clear and transparent |
| | Cycle 1 | 96.87 | 3.13 | 91.91 | With fine particles |
| | Cycle 2 | 96.68 | 3.32 | 91.66 | With fine particles |
| | Cycle 3 | 96.66 | 3.34 | 91.41 | With fine particles |
| | Cycle 5 | 96.14 | 3.86 | 91.39 | With fine particles |

### Embodiment 6

ADC-12 preparations were prepared with 10 mM succinic acid (sodium) at pH 5.3 as buffer, containing 60 mg/mL α,α-trehalose dihydrate, 0.2 mg/mL polysorbate 20 and 20 mg/mL ADC-12. The preparations were filled into a 15 mL vial with 4 mL/vail, lyophilized at a primary drying temperature of −27° C., −20° C. and −15° C., respectively, and sealed with a rubber stopper for lyophilization, followed by testing. The results indicate that −20° C. is the best primary drying temperature for the lyophilization process.

TABLE 7
Test results of ADC-12 preparations prepared through different primary drying processes

| Temperature | | | | DLS | | | | Non- |
|---|---|---|---|---|---|---|---|---|
| of primary | Water | Reconstitution | Reconstitution | Z-ave | | SEC (%) | | reduced |
| drying | content | time | appearance | (d · nm) | PDI | Monomer | Polymer | CE-SDS |
| −27° C. | 3.42% | <1 min | Clear and | 17.47 | 0.405 | 95.942 | 4.058 | 90.92 |
| | | | transparent | | | | | |
| −20° C. | 0.63% | <1 min | Clear and | 17.67 | 0.384 | 95.679 | 4.321 | 91.26 |
| | | | transparent | | | | | |
| −15° C. | 1.10% | <1 min | With | 17.18 | 0.401 | 95.685 | 4.315 | 90.45 |
| | | | crystalline | | | | | |
| | | | particles | | | | | |

### Embodiment 7

ADC-12 preparations were prepared with 10 mM succinic acid (sodium) at pH 5.3 as buffer, containing 60 mg/mL α,α-trehalose dihydrate, 0.2 mg/mL polysorbate 20 and 20 mg/mL ADC-12. The preparations were filled into glass bottle, liquid storage bag and 316L stainless steel can, respectively, and placed at 2-8° C. for 24 hours. Analysis of protein content and purity indicates (see Table 8) that ADC-12 is stable within 24 hours. The preparations are compatible with 316L stainless steel can, glass bottle and liquid storage bag. The ADC-12 preparation prepared with 10 mM succinic acid (sodium) at pH 5.3 as buffer, containing 60 mg/mL α,α-trehalose dihydrate, 0.2 mg/mL polysorbate 20 and 10 mg/mL or 1 mg/mL ADC-12 also exhibit good stability.

TABLE 8
Stability of ADC-12 in different contact materials

| | Placement | | SEC (%) | | Non-reduced | | Protein |
|---|---|---|---|---|---|---|---|
| Contact materials | temperature | Time | Monomer | Polymer | CE-SDS (%) | pH | content |
| Stainless steel | 2-8° C. | 0 | 97.65 | 2.35 | 92.21% | 5.27 | 20.72 |

| | | | | | | |
|---|---|---|---|---|---|---|
| 1 | h | 97.62 | 2.38 | 92.69% | N/A | N/A |
| 5 | h | 97.60 | 2.40 | 93.51% | N/A | N/A |
| 24 | h | 97.03 | 2.98 | 94.35% | 5.25 | 20.38 |

| | | | | | | | |
|---|---|---|---|---|---|---|---|
| Liquid storage bag | 2-8° C. | 0 | 97.65 | 2.35 | 92.21% | 5.27 | 20.72 |

| | | | | | | |
|---|---|---|---|---|---|---|
| 1 | h | 97.65 | 2.35 | 91.79% | N/A | N/A |
| 5 | h | 97.63 | 2.37 | 93.79% | N/A | N/A |
| 24 | h | 97.21 | 2.79 | 93.35% | 5.28 | 20.34 |

| | | | | | | | |
|---|---|---|---|---|---|---|---|
| Glass | 2-8° C. | 0 | 97.65 | 2.35 | 92.21% | 5.27 | 20.72 |

| | | | | | | |
|---|---|---|---|---|---|---|
| 1 | h | 97.68 | 2.32 | 91.47% | N/A | N/A |
| 5 | h | 97.64 | 2.36 | 93.42% | N/A | N/A |
| 24 | h | 97.35 | 2.65 | 92.81% | 5.26 | 20.17 |

Note:
N/A refers to not done.

### Embodiment 8: Other Alternative Formulations

The present disclosure provided stable pharmaceutical preparations comprising a combination of ADC-12 and a stabilizing buffer selected from the group consists of:

(i) 60 mg/mL α,α-trehalose dihydrate, and 10 mM succinate buffer at pH 5.3;

(ii) 60 mg/mL α,α-trehalose dihydrate, 0.2 mg/mL polysorbate 20, and 10 mM succinate buffer at pH 5.3;

(iii) 50 mg/mL α,α-trehalose dihydrate, 0.2 mg/mL polysorbate 20, and 20 mM succinate buffer at pH 5.2;

(iv) 60 mg/mL α,α-trehalose dihydrate, 0.4 mg/mL polysorbate 20, and 20 mM succinate buffer at pH 5.0;

(v) 70 mg/mL α,α-trehalose dihydrate, 0.1 mg/mL polysorbate 20, and 20 mM succinate buffer at pH 5.2;

(vi) 60 mg/mL α,α-trehalose dihydrate, 0.2 mg/mL polysorbate 20, and 10 mM succinate buffer at pH 5.2;

(vii) 60 mg/mL α,α-trehalose dihydrate, 0.4 mg/mL polysorbate 20, and 10 mM succinate buffer at pH 5.0;

(viii) 60 mg/mL α,α-trehalose dihydrate, 0.2 mg/mL polysorbate 20, and 30 mM citrate buffer pH 5.2;

(ix) 60 mg/mL α,α-trehalose dihydrate, 0.4 mg/mL polysorbate 20, and 10 mM citrate buffer at pH 5.5.

In the above embodiments, the concentration of ADC-12 ranged from 1 mg/mL to 30 mg/mL, preferably from 10 to 20 mg/mL, and most preferably 10 mg/mL. The implementable embodiments can be selected from, but not limited to, the following combinations:

(1) 30 mg/mL anti-ADC-12, 60 mg/mL α,α-trehalose dihydrate, 0.05 mg/mL polysorbate 20, and 10 mM succinate buffer at pH 5.2;

(2) 1 mg/mL anti-ADC-12, 50 mg/mL α,α-trehalose dihydrate, 0.2 mg/mL polysorbate 20, and 10 mM succinate buffer at pH 5.0;

(3) 10 mg/mL anti-ADC-12, 60 mg/mL α,α-trehalose dihydrate, 0.4 mg/mL polysorbate 20, and 10 mM succinate buffer at pH 5.1;

(4) 15 mg/mL anti-ADC-12, 50 mg/mL α,α-trehalose dihydrate, 0.3 mg/mL polysorbate 20, and 20 mM succinate buffer at pH 5.4;

(5) 5 mg/mL anti-ADC-12, 70 mg/mL α,α-trehalose dihydrate, 0.1 mg/mL polysorbate 20, and 20 mM succinate buffer at pH 5.3;

(6) 10 mg/mL anti-ADC-12, 60 mg/mL α,α-trehalose dihydrate, 0.2 mg/mL polysorbate 20, and 15 mM succinate buffer at pH 5.2;

(7) 30 mg/mL anti-ADC-12, 40 mg/mL sucrose, 0.05 mg/mL polysorbate 20, and 30 mM citrate buffer at pH 5.3;

(8) 20 mg/mL anti-ADC-12, 60 mg/mL lactose, 0.1 mg/mL polysorbate 20, and 20 mM citrate buffer at pH 5.4;

(9) 10 mg/mL anti-ADC-12, 70 mg/mL α,α-trehalose dihydrate, 0.4 mg/mL polysorbate 80, and 10 mM citrate buffer at pH 5.2;

(10) 1 mg/mL anti-ADC-12, 80 mg/mL maltose, 0.2 mg/mL polyoxyethylene hydrogenated castor oil, and 10 mM citrate buffer at pH 5.2.

While specific embodiments of the present disclosure are described above, it will be understood by those skilled in the art that they are intended only for illustration, and various changes and modifications can be made without departing from the principle and spirit of the present disclosure. Accordingly, the scope of the present disclosure is to be limited by the appended claims.

1. A pharmaceutical composition comprising a c-Met antibody drug conjugate and a buffer, wherein the buffer is a succinate buffer or a citrate buffer.

2. The pharmaceutical composition according to claim 1, wherein the concentration of the c-Met antibody drug conjugate is about 1 mg/mL to 30 mg/mL.

3. The pharmaceutical composition according to claim 1, wherein the pharmaceutical composition has a pH of about 5.0 to 6.0.

4. The pharmaceutical composition according to claim 1, wherein the buffer has a concentration of about 5 mM to 30 mM.

5. The pharmaceutical composition according to claim 1, further comprising a disaccharide, wherein the disaccharide is trehalose or sucrose.

6. The pharmaceutical composition according to claim 5, wherein the saccharide has a concentration of about 40 mg/mL to 80 mg/mL.

7. The pharmaceutical composition according to claim 1, further comprising a surfactant, wherein the surfactant is polysorbate.

8. The pharmaceutical composition according to claim 7, wherein the surfactant has a concentration of about 0.05 mg/mL to 1.0 mg/mL.

9. The pharmaceutical composition according to claim 1, comprising:
(a) 1-20 mg/mL c-Met antibody drug conjugate;
(b) 10-20 mM succinate buffer, pH 5.0-5.5;
(c) 40-80 mg/mL α,α-trehalose dihydrate; and
(d) 0.05-0.4 mg/mL polysorbate 20.

10. The pharmaceutical composition according to claim 1, wherein the c-Met antibody in the c-Met antibody drug comprises a heavy chain amino acid sequence which has at least 95% identity to a heavy chain amino acid sequence of Ab-10 antibody, and the c-Met antibody in the c-Met antibody drug conjugate comprises a light chain amino acid sequence which has at least 95% identity to a light chain amino acid sequence of Ab-10 antibody;
wherein the heavy chain amino acid sequence of Ab-10 antibody is:
QVQLVESGGGVVQPGRSLRLSCAASGFSLSNYGVHWVRQAPGKGLEWLAVI
WSGGSTNYAAAFVSRLTISKDNSKNTVYLQMNSLRAEDTAVYYCARNHDNP
YNYAMDYWGQGTTVTVSSASTKGPSVFPLAPCSRSTSESTAALGCLVKDYF
PEPVTVSWNSGALTSGVHTFPAVLQSSGLYSLSSVVTVPSSNFGTQTYTCN
VDHKPSNTKVDKTVERKCCVECPPCPAPPVAGPSVFLEPPKPKDTLMISRT
PEVTCVVVDVSHEDPEVQFNWYVDGVEVHNAKTKPREEQFNSTFRVVSVLT
VVHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYSK
LTVDKSRWQQGNVFSCSVMHEALHNHYTQKSLSLSPGK
and wherein the light chain amino acid sequence of Ab-10 antibody is:
DIVLTQSPDSLAVSLGERATINCRADKSVSTSTYNYLHWYQQKPGQPPKLL
IYLASNLASGVPDRFSGSGSGTDFTLTISSLQAEDVAVYYCQHSRDLPPTF
GQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC.

11. The pharmaceutical composition according to claim 1, wherein the c-Met antibody drug conjugate is ADC-12, which has a structure shown as follows: 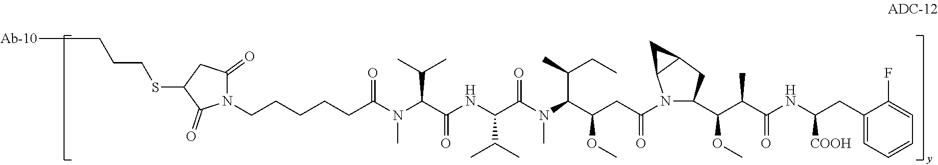
wherein y ranges from 1 to 8.

12. A method of preparing a lyophilized preparation comprising c-Met antibody drug conjugate, the method comprising lyophilizing the pharmaceutical composition according to claim 1.

13. The method according to claim 12, wherein, the lyophilizing comprises prefreezing pre-freezing, primary drying and secondary drying in sequence.

14. A lyophilized preparation comprising c-Met antibody drug conjugate prepared by the method according to claim 12.

15. A lyophilized preparation reconstituted to form the pharmaceutical composition according to claim 1.

16. (canceled)

17. A method of treating and preventing a disease or condition associated with c-Met, comprising administering a therapeutically effective amount of the pharmaceutical composition according to claim 1 to a subject in need thereof, wherein the disease or condition is a cancer.

18. A product comprising a container, wherein the container comprises the pharmaceutical composition according to claim 1.

19. The method according to claim 17, wherein the cancer is a c-Met-expressing cancer, and the c-Met-expressing cancer is selected from the group consisting of c-Met-expressing gastric cancer, pancreatic cancer, lung cancer, glioblastoma, sarcoma, colorectal cancer, renal cancer, hepatocellular carcinoma, melanoma and breast cancer.

20. A pharmaceutical composition comprising:
(a) 1-20 mg/mL c-Met antibody drug conjugate;
(b) 10-20 mM succinate buffer, pH 5.0-5.5;
(c) 60 mg/mL α,α-trehalose dihydrate; and
(d) 0.05-0.4 mg/mL polysorbate 20.

21. A pharmaceutical composition comprising:
(a) 5-20 mg/mL c-Met antibody drug conjugate;
(b) 10-20 mM succinate buffer, pH 5.0-5.5;
(c) 50-70 mg/mL α,α-trehalose dihydrate; and
(d) 0.1-0.2 mg/mL polysorbate 20.
